(19) United States
(12) Patent Application Publication
(10) Pub. No.: US 2010/0016726 A1
(43) Pub. Date: Jan. 21, 2010

(54) Handheld Imaging Device And Method For Manufacture Thereof

(76) Inventor: Joseph H. Meier, McKinney, TX (US)

Correspondence Address: BRYAN CAVE LLP, TWO NORTH CENTRAL AVENUE, SUITE 2200, PHOENIX, AZ, 85004, US

(21) Appl. No.: 12/176,194

(22) Filed: Jul. 18, 2008

(51) Int. Cl. A61B 8/14

(52) U.S. Cl. 600/459; 29/592.1

(57) ABSTRACT

In one embodiment, an ultrasound imaging device is configured to facilitate sub-dermal monitoring. The ultrasound imaging device comprises a handheld housing, a processor within the handheld housing, a beamformer coupled to the processor, a transducer assembly coupled to the handheld housing and to at least one of the beamformer and the processor, a scan converter coupled to the transducer assembly, a display coupled to the handheld housing and coupled to at least one of the scan converter and the processor, a switch mechanism coupled to the processor, a rechargeable power source coupled to the handheld housing, a communications port coupled to the processor, a central pointer aligned with a center of the display, and a needle guide coupled to the handheld housing proximate to the transducer assembly. Other examples and embodiments are described herein.

### TECHNICAL FIELD

This disclosure relates generally to imaging devices, and relates more particularly to handheld imaging devices and methods of manufacture for handheld imaging devices.

### BACKGROUND

The use of non-invasive monitoring systems, such as ultrasound devices, to produce real-time images of blood vessels, organs, bones, nerves, tumors, and other target structures under the skin or other layers of tissue in patients has advanced the techniques used for interacting with such target structures. Procedures for epidural placements, lumbar punctures, nerve blockings, and the cannulation of vascular vessels, among other procedures, have been accordingly advanced. For example, prior to the development of such systems, medical practitioners attempting to cannulate a vascular vessel had to rely on approximations of the predicted locations of such target structures, without any internal visual aids to guide the cannulation process through the interior of the patient. This cannulation technique can produce unwanted results, such as the puncturing of wrong vascular vessels or structures, and/or repeated painful attempts to locate and cannulate the correct structure.

Although technology has advanced the monitoring process, cannulation still requires hand/eye coordination between the images scanned by a monitoring system and a needle or probe as it is inserted by the hand of the medical practitioner into a target area of a patient. Accordingly, a need exists for a monitoring device that can present real-time internal images of the cannulation process proximate to, and aligned with, the target area and internal target structure to, therefore, assist the hand/eye coordination of the medical practitioner during the monitoring and/or cannulation process.

### BRIEF DESCRIPTION OF THE DRAWINGS

The invention will be better understood from a reading of the following detailed description of examples of embodiments, taken in conjunction with the accompanying figures in the drawings in which:

FIG. 1 illustrates a block diagram of a monitoring device.

FIG. 2 illustrates a front perspective view of the monitoring device of FIG. 1.

FIG. 3 illustrates a bottom view of the monitoring device of FIG. 1.

FIG. 4 illustrates a bottom view of a transducer for the monitoring device of FIG. 1.

FIG. 5 illustrates a bottom view of another transducer for the monitoring device of FIG. 1.

FIG. 6 illustrates a side view of another monitoring device.

FIG. 7 illustrates a side view of a different monitoring device.

FIG. 8 illustrates a side view of the monitoring device of FIG. 1 partially covered by a casing.

FIG. 9 illustrates a cross-sectional side view of another casing configured to cover the monitoring device of FIG. 6.

FIG. 10 illustrates a cross-sectional side view of a different casing configured to cover the monitoring device of FIG. 7.

FIG. 11 illustrates a perspective view of yet another monitoring device.

FIG. 12 illustrates a block diagram of the monitoring device of FIG. 11.

FIG. 13 illustrates a block diagram of a method of manufacturing a handheld imaging device similar to the monitoring devices of FIGS. 1-11.

For simplicity and clarity of illustration, the drawing figures illustrate the general manner of construction, and descriptions and details of well-known features and techniques may be omitted to avoid unnecessarily obscuring the invention. Additionally, elements in the drawing figures are not necessarily drawn to scale. For example, the dimensions of some of the elements in the figures may be exaggerated relative to other elements to help improve understanding of examples of embodiments. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments of the invention described herein are, for example, capable of operation in sequences other than those illustrated or otherwise described herein. Furthermore, the terms “include,” and “have,” and any variations thereof, are intended to cover a non-exclusive inclusion, such that a process, method, article, or apparatus that comprises a list of elements is not necessarily limited to those elements, but may include other elements not expressly listed or inherent to such process, method, article, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the invention described herein are, for example, capable of operation in other orientations than those illustrated or otherwise described herein. The term “coupled,” as used herein, is defined as directly or indirectly connected in an electrical, physical, mechanical, or other manner.

### DETAILED DESCRIPTION OF EXEMPLARY EMBODIMENTS

In one embodiment, an ultrasound imaging device is configured to facilitate sub-dermal monitoring. The ultrasound imaging device comprises a handheld housing, a processor within the handheld housing, a beamformer coupled to the processor, a transducer assembly coupled to the handheld housing and to at least one of the beamformer and the processor, a scan converter coupled to the transducer assembly, a display coupled to the handheld housing and coupled to at least one of the scan converter and the processor, a switch mechanism coupled to the processor, a rechargeable power source coupled to the handheld housing, a communications port coupled to the processor, a central pointer aligned with a center of the display, and a needle guide coupled to the handheld housing proximate to the transducer assembly. The transducer assembly comprises a first transducer array coupled to the processor and aligned along a first axis, and a second transducer array coupled to the processor and aligned along a second axis different from the first axis, where the first transducer array and the second transducer array are configured to produce an overlapping scan at a target focus point. The first axis is longitudinal to the target focus point; the second axis is transverse to the target focus point; and the first transducer array and the second transducer array are substantially perpendicular to each other. The first transducer array comprises transducer elements configured to scan images along the first axis, and the second transducer array comprises transducer elements configured to scan images along the second axis. The first and second transducer arrays are capable of concurrently imaging the target focus point. The rechargeable power source is cordless and configured to power the monitoring device uninterrupted for at least approximately a half-hour, and the ultrasound imaging device is configured for single-handed operation.

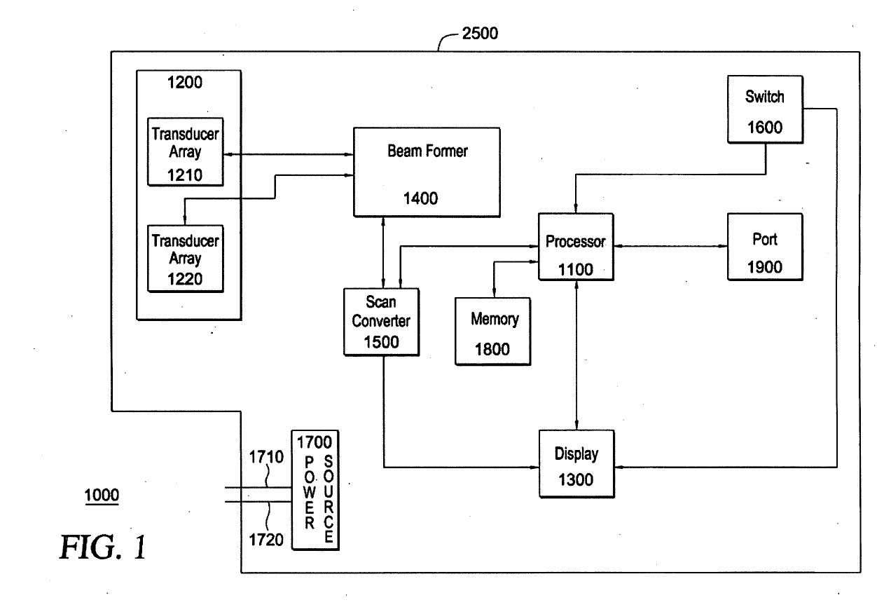

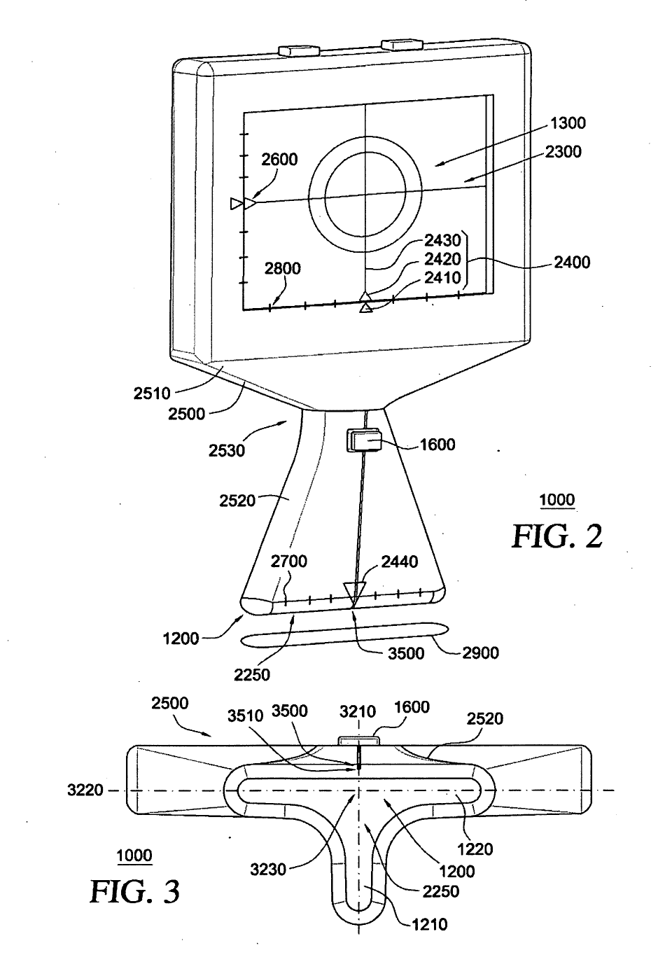

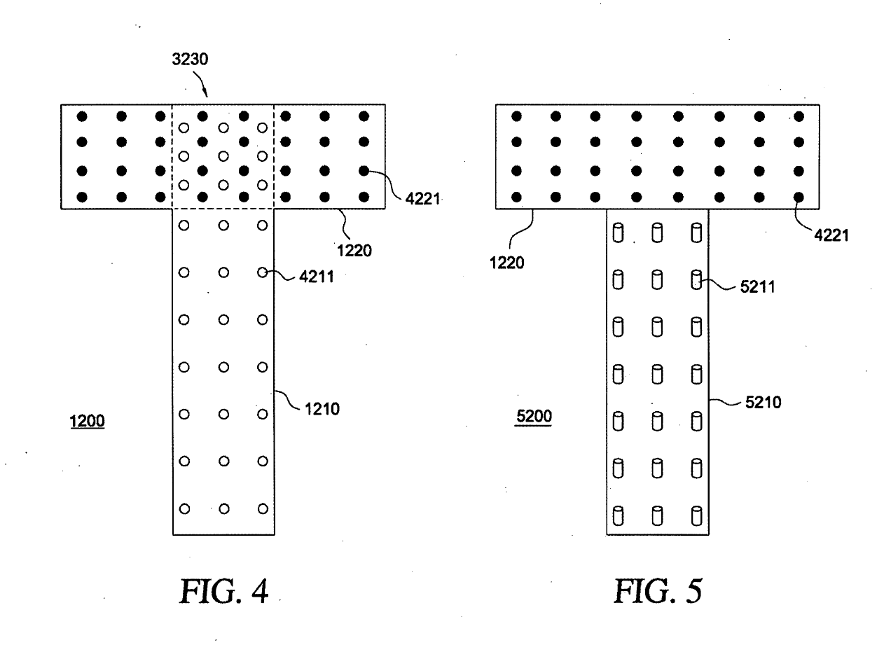

Turning over to the figures, FIG. 1 illustrates a block diagram of a monitoring device 1000. FIG. 2 illustrates a front perspective view of monitoring device 1000. FIG. 3 illustrates a bottom view of monitoring device 1000. FIG. 4 illustrates a bottom view of transducer 1200 of monitoring device 1000. FIG. 5 illustrates a bottom view of transducer 5200 of monitoring device 1000.

In some embodiments, monitoring device 1000 can be used in the medical field for intra-tissue or sub-dermal inspection on a patient. As an example, monitoring device 1000 can be used to facilitate non-invasive imaging of vascular vessels, such as veins and arteries, through skin and/or other tissue. In one example, such imaging can be useful to guide a medical practitioner while cannulating a vascular vessel, allowing the medical practitioner to align, position, and guide a needle into the vascular vessel. In some embodiments, the needle can comprise a probe and/or a catheter.

In the present embodiment, monitoring device 1000 comprises processor 1100 within housing 2500. Processor 1100 can comprise, for example, a microprocessor such as a general microprocessor for personal computers, and/or a specialized microprocessor for a specific implementation such as analog and mixed signal operations. Monitoring device 1000 can also comprise memory 1800 coupled to processor 1100. Memory 1800 can be used to store software instructions for operating monitoring device 1000, and/or information such as images scanned using monitoring device 1000. In the same or a different embodiment, memory 1800 can comprise non-volatile memory, such as flash memory, and/or magnetic storage such as hard disks. In some embodiments, memory 1800 can comprise removable memory devices, such as SD (Secure Digital) cards. In a different embodiment, processor 1100 and memory 1800 can be combined to form a microcontroller.

Monitoring device 1000 also comprises display 1300 coupled to processor 1100 and to housing 2500. In some embodiments, display 1300 can comprise a width of approximately 3 to 8 centimeters, and/or a height of approximately 2 to 5 centimeters. In one embodiment, display 1300 can comprise at least one of a Liquid Crystal Display (LCD), a touch-screen display, and a Thin Film Transistor (TFT) display. In the same or a different embodiment, display 1300 can be configured to present a data entry screen, where information such as a patient's name and/or a medical record number can be entered by interacting with the data entry screen. In the same or a different embodiment, the data entry screen can be configured to accept input from a touch-screen coupled to display 1300, a keypad coupled to monitoring device 1000, and/or a point and click mechanism. In the same or a different embodiment, information entered into the data entry screen can be stored into memory 1800, and/or can be correlated to information or images stored in memory 1800.

Display 1300 can be configured to be aligned with, and visible through, translucent portion 2300 (FIG. 2) of housing 2500, where translucent portion 2300 can comprise a translucent material, transparent material, or a cutout. In some embodiments, display 1300 can also be coupled to a graphics adapter (not shown). In the same or a different embodiment, the graphics adapter can be part of processor 1100. In a different embodiment, monitoring device 1000 can also comprise additional displays similar to display 1300.

Monitoring device 1000 also comprises transducer 1200 coupled to processor 1100 and to housing 2500. In the present embodiment, transducer 1200 comprises transducer arrays 1210 and 1220 coupled to processor 1100, where transducer array 1210 is aligned along axis 3210 (FIG. 3), and where transducer array 1220 is aligned along axis 3220 (FIG. 3) of scanning surface 2250 (FIG. 2). In the same or a different embodiment, transducer arrays 1210 and 1220 can be ultrasonic transducer arrays comprising piezoelectric elements configured to emit ultrasonic beams and/or to detect reflections of the ultrasonic beams.

Transducer arrays 1210 and 1220 are configured to produce different but overlapping scans (not shown), where an image can be presented on display 1300 based on readings from the overlapping scan. In the present embodiment, as more clearly seen in FIG. 4, transducer arrays 1210 and 1220 overlap at transducer junction 3230 of transducer 1200, where respective elements 4211 and 4221 of transducer arrays 1210 and 1220 coincide. In the same or a different embodiment one or more of elements 4211 and 4221 that are proximate to transducer junction 3230 can be shared by both transducer arrays 1210 and 1220. Portions of transducer arrays 1210 and 1220 overlap substantially perpendicular to each other. In addition, beams emitted by elements 4211 and 4221 are substantially perpendicular to scanning surface 2250. As also seen in FIG. 4, other portions of transducer arrays 1210 and 1220 do not overlap with each other.

In a different embodiment, as more clearly seen in FIG. 5, monitoring device 1000 can comprise transducer 5200 with transducer arrays 1210 and 5220. Transducer 5200 can also produce different but overlapping scans similar to the overlapping scans produced by transducer 1200, but transducer 5200 differs in that there is no transducer junction because transducer arrays 1210 and 5220 do not physically overlap. Instead, transducer array 5210 comprises elements 5211 that are configured to transmit and/or detect ultrasonic beams at an angle such as to overlap with beams transmitted by elements 4221 of transducer array 1220. In a different embodiment, elements 4221 are modified to transmit and/or detect ultrasonic beams at an angle to overlap with beams transmitted by elements 5211, which are modified to transmit beams substantially perpendicular to scanning surface 2250. In another embodiment, both elements 4221 and 5211 are angled.

Returning to the embodiment of FIGS. 1-4, transducer 1200 is at least partially enclosed by housing 2500. Similarly, display 1300 is at least partially enclosed by housing 2500. In the same or a different embodiment, transducer 1200 and display 1300 can be integrated with housing 2500 such as to form a single handheld unit out of monitoring device 1000, with no external cables to interconnect display 1300 and/or transducer 1200 to housing 2500 and/or processor 1100. In the same or a different embodiment, housing 2500 can comprise materials such as metal, acrylics, polycarbonates, and other rigid or semi-rigid plastics. As used herein, the term “integrated” allows for interchangeable portions of monitoring device 1000. For example, in the same or a different embodiment, housing 2500 can be integrated with a different transducer, such as transducer 5200, which is replaceable or interchangeable with transducer 1200.

In the present embodiment, as shown in FIG. 1, monitoring device 1000 comprises beamformer 1400 coupled to transducer 1200. In addition, processor 1100 couples to transducer 1200 through beamformer 1400. In the present example, beamformer 1400 is configured to control the timing, strength, angle, amplitude, and/or phase of ultrasound signals transmitted by transducer arrays 1210 and 1220. In the same or a different example, beamformer 1400 can be configured to control transducer arrays 1210 and 1220 to receive signals predominantly from a chosen angular direction.

Continuing with the present embodiment, monitoring device 1000 also comprises scan converter 1500 coupled to beamformer 1400 and to display 1300. In some embodiments, scan converter 1500 can be coupled to display 1300 via processor 1100. Scan converter 1500 can be used to convert information from ultrasound signals received by transducer arrays 1210 and 1220 into an image format that can be displayed on, for example, display 1300. In the present embodiment, beamformer 1400 can comprise at least one of a B-mode, F-mode, and a D-mode acquisition mode.

As described above, monitoring device 1000 can be used to image through target location 2900 (FIG. 2), where target location 2900 can be under the skin surface of a patient or person. In the present embodiment, axis 3210 (FIG. 3) is longitudinal to target location 2900, while axis 3220 is transverse to target location 2900. In addition, display 1300 is substantially parallel to axis 3220. Transducer 1200 is configured to scan a set of readings of target location 2900 using at least a portion of transducer array 1210, while simultaneously scanning a different set of readings of target location 2900 using at least a portion of transducer array 1220. In the current embodiment, at least one of transducer arrays 1210 and 1220 is configured to scan a depth of field of up to approximately 10 centimeters.

In the same or a different embodiment, at least one of transducer arrays 1210 and 1220 is configured to scan a span of up to approximately 4 to 5 cm. In the same or a different embodiment, at least one of transducer arrays 1210 and 1220 can be configured to scan at a transducer frequency of approximately between 2 and 50 MHz.

In the present embodiment, monitoring device 1000 also comprises a switch mechanism 1600 coupled to processor 1100. Switch Mechanism 1600 is configured to deactivate transducer array 1220 and activate transducer array 1210 in response to a first setting of switch mechanism 1600. In addition, switch mechanism 1600 is configured to deactivate transducer array 1210 and activate transducer array 1220 in response to a second setting of switch mechanism 1600. In the present embodiment, the settings of switch mechanism 1600 are recognized by processor 1000, which causes transducer arrays 1210 and/or 1220 to activate or deactivate accordingly and which changes the image(s) shown on display 1300. In a different embodiment, switch mechanism 1600 can communicate more directly with transducer arrays 1210 and/or 1220, such as through beamformer 1400, to activate or deactivate transducer arrays 1210 and/or 1220 accordingly.

In the same or a different embodiment, monitoring device 1000 is configured for one-handed operation. For example, monitoring device 1000 can be configured to allow a hand to grab around portion 2520 (FIG. 2) of housing 2500, such that switch mechanism 1600 can be still operable by a finger (e.g., a thumb) of the same hand without releasing portion 2520. In addition, monitoring device 1000 can be configured for non-dominant handed operation. Such non-dominant handed configuration can be advantageous, for example, to free-up a user's dominant hand to cannulate a vascular vessel monitored through monitoring device 1000. In some embodiments, a weight of monitoring device 1000 is between approximately 0.3 and 0.7 kilograms.

In some embodiments, monitoring device 1000 can comprise other switches or buttons to control other operations or features of monitoring device 1000. Such other switches can comprise one or more of an on/off control, a gain control, a depth control, a focus control, a brightness control, and/or a contrast control.

Display 1300, in the current embodiment of monitoring device 1000, is configured to present images correlated to readings from transducer array 1210 in response to one setting of switch mechanism 1600. Display 1300 is also configured to present images correlated to a set of readings from transducer array 1220 in response to a different setting of switch mechanism 1600. In the present embodiment, because display 1300 is sized to allow monitoring device 1000 to be handheld, it can be clearer for display 1300 to present images from only one of transducer arrays 1210 and 1220 at a time. Switch mechanism 1600 can therefore be used to toggle the source of images on display 1300 from array 1210 to 1220, and vice-versa. In a different embodiment, however, monitoring device 1000 can be configured to simultaneously present images correlated to readings from transducer array 1210 on one portion of display 1300, and images correlated to readings from transducer array 1220 on another portion of display 1300.

In the same or a different embodiment, display 1300 can also present other information, such as menu screens and/or other images. In the same or a different embodiment, switch mechanism 1600 can also be used to toggle display 1300 to and from presenting such other information. In a different embodiment, switch mechanism 1600 can comprise more than one switch, where different switches can be correlated to additional displays similar to display 1300, and/or to individual transducer arrays similar to transducer arrays 1210 and 1220.

FIG. 1. illustrates power source 1700. In the present embodiment, power source 1700 comprises a portable battery, which can be rechargeable. Power source 1700 is coupled to housing 2500, and is configured to power electrical systems of monitoring device 1000, such as processor 1100 and transducer 1200, among others. In the present embodiment, power source 1700 is located within housing 2500. In a different embodiment, power source 1700 can be attached to an exterior surface of housing 2500. In a different embodiment, power source 1700 can comprise a power cord to recharge power source 1700, where the power cord can be detachable in some examples.

In some embodiments, power source 1700 can be configured to be charged via a docking station (not shown), where the docking station can be tailored accommodate and/or support a portion of the surface of housing 2500. In one embodiment, power source 1700 comprises charging leads 1711-1712 accessible through the exterior of housing 2500, and the docking station comprises contact leads (not shown) complementary with charging leads 1711-1712. The contact leads in the same embodiment can be configured to contact charging leads 1711-1712 to charge power source 1700 when monitoring device 1000 is docked with the docking station. In a different embodiment, the docking station can be configured to charge power source 1700 via one of a capacitive coupling or an inductive coupling, where direct contact between charging and/or contact leads may not be needed.

As shown in FIG. 1, the present embodiment also comprises port 1900 coupled to processor 1100. Port 1900 can be used to place monitoring device 1000 in communication with other electronic devices. For example, port 1900 can be used to interface monitoring device 1000 with a personal computer or a database to transmit information such as scanned images. In one example, port 1900 can comprise a wired port, such as a USB or Firewire® port. In the same or a different example, port 1900 can also comprise a wireless port. In some examples, the docking station described above for power source 1700 can also be configured to couple with port 1900 to facilitate the communication with other electronic devices when monitoring device 1000 is docked with the docking station.

As more clearly illustrated in FIG. 2, monitoring device 1000 comprises a central pointer 2400 configured to indicate a center of an image shown on display 1300. In some embodiments, central pointer 2400 comprises one or more of pointer 2410 on housing 2500, pointer 2420 presented on display 1300, pointer line 2430 presented also on display 1300, and/or pointer 2440 proximate to scanning surface 2250. In the present example, central pointer 2400 indicates a midpoint of display 1300. Central pointer 2400 is correlated to a centerline of transducer array 1210 (FIGS. 1, 3, and 4) in the current example. In the present or a different example, monitoring device 1000 can comprise central pointer 2600, similar to central pointer 2400, but correlated instead to a centerline of transducer array 1220 (FIGS. 1, 3, and 4). In the present embodiment, the centerlines of transducer arrays 1210 and 1220 can correspond to axes 3210 and 3220, respectively, in FIG. 3.

In the present embodiment, housing 2500 comprises gridmarks 2700 aligned along an axis substantially parallel to axis 3220. In addition, monitoring device 1000 comprises grid pointers 2800 configured to demarcate on display 1300 subdivisions correlated to gridmarks 2700. Grid pointers 2800 can comprise physical and/or electronic grid pointers.

As seen in FIG. 3, monitoring device 1000 comprises needle guide 3500 aligned with transducer array 1210 and proximate to a central portion of transducer array 1220. In the present example, needle guide 3500 is coupled to housing 2500 proximate to scanning surface 2250. Needle guide 3500 is substantially in-line with axis 3210 in the present example, and comprises needle alignment groove 3510. In one embodiment, needle guide 3500 can be used to assist a user in aligning a needle with central pointer 2400 prior to and during cannulation of a vascular vessel presented on display 1300.

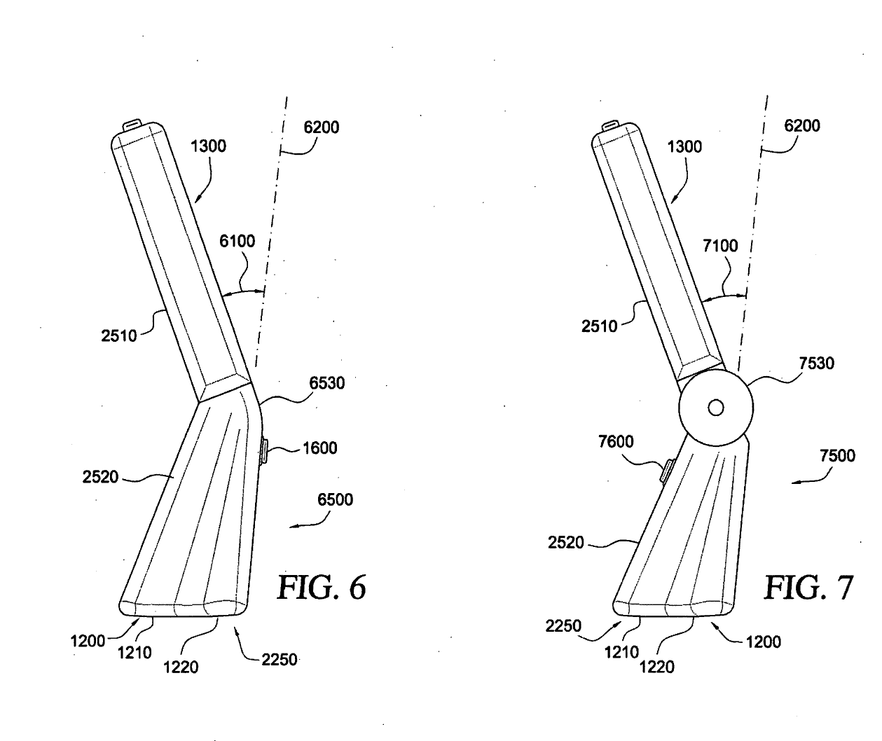

Continuing with the figures, FIG. 6 illustrates a side view of monitoring device 6000. FIG. 7 illustrates a side view of monitoring device 7000.

Monitoring devices 6000 and 7000 are similar to monitoring device 1000 (FIGS. 1-6), but differ by comprising housings 6500 and 7500, respectively, similar to housing 2500 (FIG. 2). Housing 6500 in FIG. 6 comprises joint 6530 between portions 2510 and 2520 of housing 6500. Similarly, housing 7500 in FIG. 7 comprises joint 7530 between portions 2510 and 2520 of housing 7500. In contrast with joint 2530 of housing 2500 (FIG. 2), where portions 2510 and 2520 of housing 2500 are substantially planar relative to each other, joints 6530 and 7530 permit their respective portions 2510 and 2520 to be angled relative to each other to facilitate viewing of display 1300.

FIG. 6 shows portion 2510 is fixedly angled towards a rear of monitoring device 6000 at angle 6100. In the present example, angle 6100 comprises approximately 25 degrees. In some examples, angle 6100 can be fixed at approximately between 10 to 45 degrees. In the example of FIG. 7, joint 7530 permits portion 2510 to be variably angled and adjustable relative to portion 2520 of housing 7500. In some embodiments, angle 7100 can be varied between approximately 0 and 90 degrees. In some examples, display 1300 can also be rotated about axis 6200.

In the example of FIG. 7, monitoring device 7000 comprises switch mechanism 7600, similar to switch mechanism 1600 of FIGS. 1-3 and 6. Switch mechanism 7600 differs by being located towards the rear of monitoring device 7000 such as to be operable in a pistol-trigger fashion. This arrangement could facilitate the single-handed operation of monitoring device 7000 when portion 2520 of housing 7500 is grabbed by a hand.

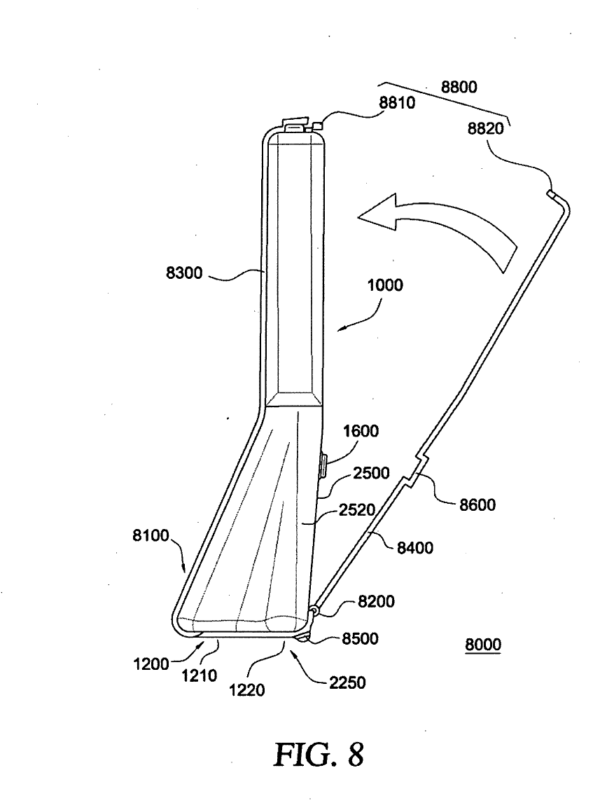

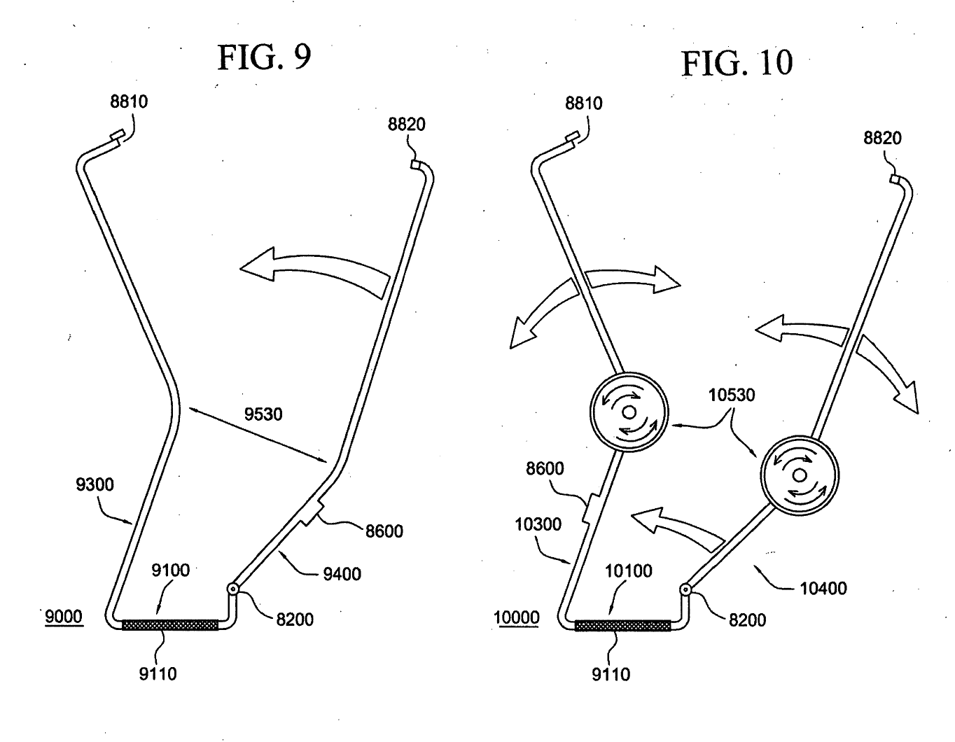

Moving on, FIG. 8 illustrates a side view of monitoring device 1000 (FIGS. 1-5) partially covered by casing 8000. FIG. 9 illustrates a cross-sectional side view of casing 9000 configured to cover monitoring device 6000 (FIG. 6). FIG. 10 illustrates a cross-sectional side view of casing 10000 configured to cover monitoring device 7000 (FIG. 7). In some embodiments, at least one of casings 8000, 9000, and/or 10000 can be referred to as a cover.

Casing 8000 comprises transducer cover portion 8100, configured to removably envelop at least a portion of housing 2500. In the present embodiment, transducer arrays 1210 and 1220 are arranged in a T-shape, as shown in FIG. 4, and portion 8100 is configured to accommodate the T-shape. Casing 8000 comprises hinge 8200 to permit portions 8300 and 8400 to envelop monitoring device 1000 in a clamshell fashion. Transducer cover portion 8100 is transparent proximate to scanning surface 2250 with respect to transducer arrays 1210 and 1220 such as to minimize interference with the transmission and reception of signals from transducer 1200.

In the present example, transducer cover portion 8100 also comprises needle guide 8500, similar to needle guide 3500 as described above for FIG. 3. Casing 8000 is configured to be disposable and/or sterilizable, such that monitoring device 1000 can be brought into and used at a clean room or sterile environment.

Casings 9000 and 10000 are similar to casing 8000, but differ by allowing for an angle between portions 2510 and 2520 of monitoring devices 6000 and 7000. Similar to casing 8000, casings 9000 and 10000 also comprise hinge 8200 to permit portions 9300 and 9400, and portions 10300 and 10400, respectively, to envelop monitoring devices 6000 and 7000 in a clamshell fashion. In the embodiments of FIGS. 8-10, locks 8810 and 8820 of locking mechanism 8800 can be brought together to secure casings 8000, 9000, and 10000 when closed.

In some embodiments, one or more portions of casings 8000, 9000, and/or 10000 can comprise materials such as rigid plastic, semi-rigid plastic, and/or flexible materials such as silicone. In the same or a different embodiment, at least a portion of casings 8000, 9000, and/or 10000 can conform to a shape of a portion of monitoring device 1000, 6000, and/or 7000. As an example, portion 9530 of casing 9000 in FIG. 9 and portion 10530 of casing 10000 in FIG. 10 can comprise a semi-rigid or flexible material to accommodate the envelopment of joint 6530 (FIG. 6) or joint 7530 (FIG. 7). In the same or a different embodiment, portion 9530 of casing 9000 and portion 10530 of casing 10000 can be configured in an accordion manner to allow for the angle between portions 2510 and 2520. In some embodiments, switch mechanisms 1600 (FIGS. 1, 6, 8) and 7600 (FIG. 7) of respective monitoring devices 1000, 6000, and 7000 can be covered by pliable portions 8600 of casings 8000, 9000, and 10000, respectively. In some examples, pliable portions 8600 can comprise materials similar to those materials described above for portion 9530, and can permit a user to operate switch mechanisms 1600 and 6600 while covered by casings 8000, 9000, and 10000.

The embodiments shown in FIGS. 9-10 show transducer casings 9100 and 10100 comprising gel-pack 9110 positioned proximate to transducer arrays 1210 and 1220 (FIGS. 6-7). In some embodiments, gel-pack 9110 can comprise a bladder filled with an aqueous, flexible gel material suitable for the transmission of ultrasound signals. In the same or a different embodiment, gel-pack 9110 can be similar to an Aquaflex® gel pad from Parker Laboratories, Inc. In a different embodiment, casing 8000 can also comprise gel-pack 9110.

In some embodiments, part of transducer cover portion 8100 can comprise a non-stick material proximate to scanning surface 2250 to facilitate sliding monitoring device 1000 over a target surface. In the same or a different embodiment, one or more of transducer cover portions 8100, 9100, and/or 10100 can comprise a T-shape tailored to dimensions of transducer arrays 1210 and 1220 on portion 2520 (FIGS. 34). In the some examples, a thickness of a portion of one or more of casings 8000, 9000, and/or 10000 comprises approximately between 0.5 to 5 millimeters. In a embodiment different than as illustrated in FIGS. 8-10, a cover similar to casing 8000 can be configured to leave display 1300 exposed so as to cover only, for example, portion 2520 of monitoring device 1000.

In some embodiments, monitoring devices 6000 and 7000 can be charged via a docking station (not shown), similar to as described above for monitoring device 1000. In the same or a different example, the docking station can also be configured to charge power source 1700 while monitoring devices 1000, 6000, and/or 7000 are covered by casings 8000, 9000, and 10000, respectively.

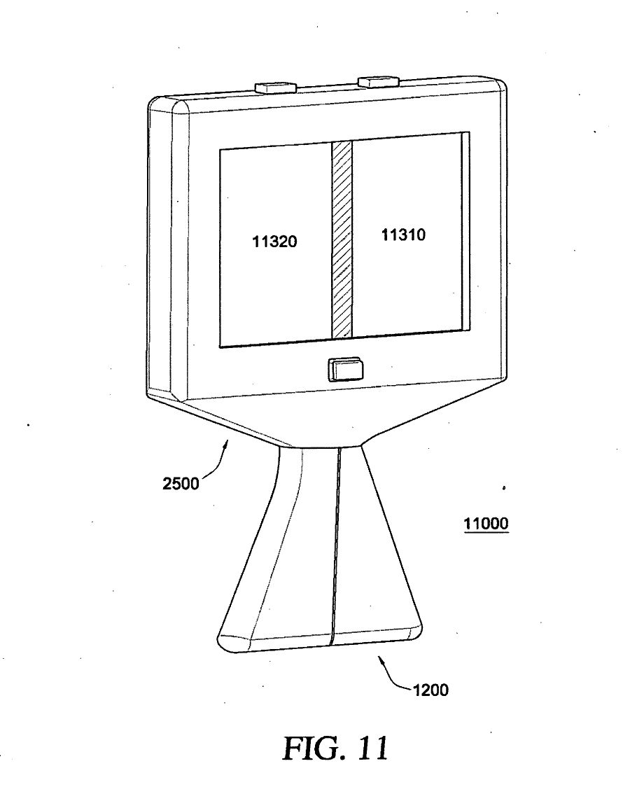

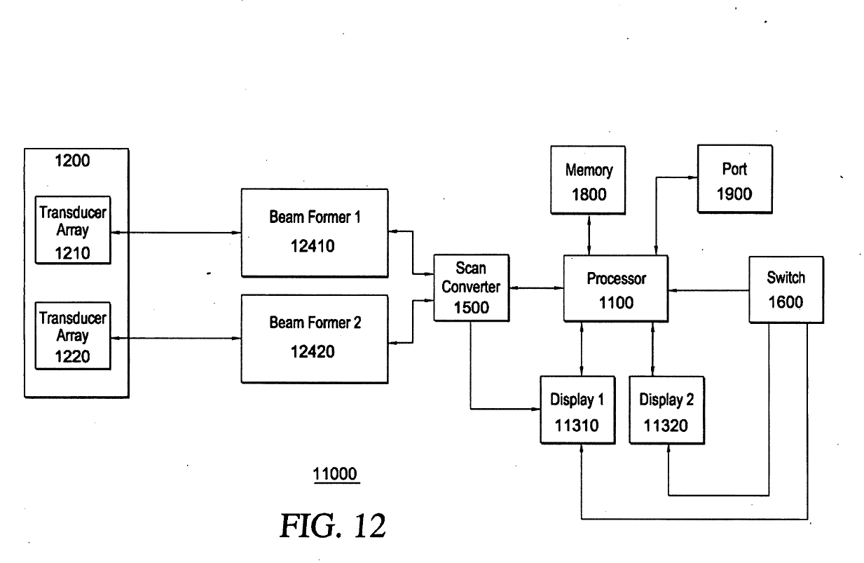

Continuing with the figures, FIG. 11 illustrates a perspective view of a monitoring device 11000. FIG. 12 illustrates a block diagram of monitoring device 11000. Monitoring device 11000 is similar to monitoring device 1000, but comprises displays 11310 and 11310 rather than a single display. Monitoring device 11000 comprises transducer 1200 like monitoring device 1000, and is configured to simultaneously present images correlated to readings from transducer array 1210 on display 11310, and images correlated to readings from transducer array 1220 on display 11320. As seen in FIG. 12, monitoring device 11000 comprises beamformers 12410 and 12420 configured to control and couple to transducer arrays 1210 and 1220, respectively. In the present embodiment, beamformers 12410 and 12420 connect to scan converter 1500, although in a different embodiment beamformers 12410 and 12420 can connect to their own dedicated scan converters.

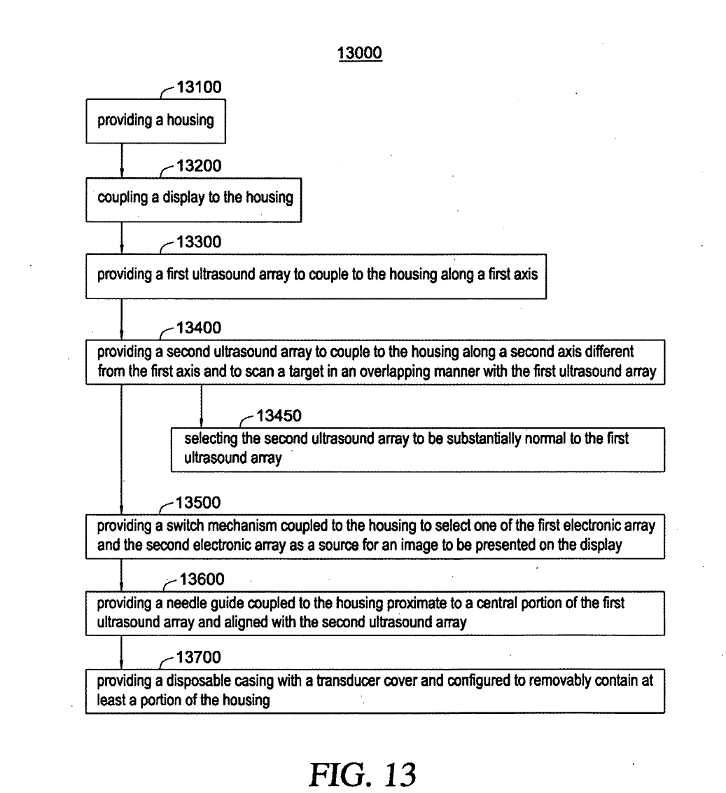

Moving on, FIG. 13 illustrates a block diagram of a method of manufacturing a handheld imaging device. In some embodiments, the handheld imaging device can be one of monitoring devices 1000 (FIGS. 1-3, 8), 6000 (FIG. 6), 7000 (FIG. 7), and 11000 (FIGS. 11-12).

Block 13100 of method 13000 comprises providing a housing. In one example, the housing can be one of housings 2500 (FIGS. 2, 3, 8, 11), 6500 (FIG. 6), 7500 (FIG. 7).

Block 13200 of method 13000 comprises coupling a display to the housing of block 13100. In some examples, the display can be similar to the display described above for display 1300 (FIGS. 1-2), 113310, and 11320 (FIGS. 11-12).

Block 13300 of method 13000 comprises providing a first ultrasound array to couple to the housing of block 13100 along a first axis. In some examples, the first ultrasound array can be similar to the array described above for transducer arrays 1210 (FIGS. 1, 3, 4) and 5210 (FIG. 5). Similarly, the first axis can be similar to axis 3210 in FIG. 3.

Block 13400 of method 13000 comprises providing a second ultrasound array to couple to the housing of block 13100 along a second axis different from the first axis of block 13300, and to scan a target in an overlapping manner with the first ultrasound array of block 13300. In one embodiment the second ultrasound array can be similar to the array described above for transducer array 1220 (FIGS. 1, 3, 4) and 5220 (FIG. 5). Similarly, the second axis can be similar to axis 3220 in FIG. 3. In one embodiment, the first and second ultrasound arrays of blocks 13300 and 13400 can scan the target in overlapping manner by overlapping as shown and described for transducer arrays 1210 and 1220 in FIG. 4. In a different embodiment, the first and second ultrasound arrays can scan the target in overlapping manner as shown and described for transducer arrays 1210 and 5220 in FIG. 5.

Block 13450 of method 13000 comprises selecting the second ultrasound array of block 13400 to be substantially normal to the first ultrasound array of block 13300. Block 13450 can be a sub-part of block 13400. In some examples, the second ultrasound array of block 13400 can be substantially normal to the first ultrasound array of block 13300 as shown in FIG. 4 for transducer arrays 1210 and 1220, or as shown in FIG. 5 for transducer arrays 5120 and 5220.

Block 13500 of method 13000 comprises providing a switch mechanism coupled to the housing of block 13100 to select one of the first electronic array of block 13300 and the second electronic array of block 13400 as a source for an image to be presented on the display of block 13200. In one embodiment, the switch mechanism can be similar to switch mechanism 1600 (FIGS. 1-3). In the same or a different example, the switch mechanism can be configured to deactivate the second ultrasound array of block 13400 and activate the first ultrasound array of block 13300 in response to a first setting of the switch mechanism, and to deactivate the first ultrasound array of block 13300 and activate the second ultrasound array of block 13400 in response to a second setting of the switch mechanism. In the same or a different example, the display of block 13200 is configured to present images scanned from the first ultrasound array of block 13300 in response to the first setting of the switch mechanism, and to present images scanned from the second ultrasound array of block 13400 in response to the second setting of the switch mechanism.

Block 13600 of method 13000 comprises providing a needle guide aligned with the first transducer array of block 13300 and proximate to a central portion of the second transducer array of block 13400. In some examples, the needle guide can be similar to the guide described for needle guide 3500 (FIG. 3).

Block 13700 of method 13000 comprises providing a disposable casing with a transducer cover and configured to removably contain at least a portion of the housing of block 13100. In some examples, the disposable casing can be as described above for casings 8000, 9000, and/or 10000 (FIGS. 8-10).

In some embodiments, the sequence of blocks 13100, 13200, 13300, 13400, 13450, 13500, 13600, and/or 13700 of method 13000 can be changed or otherwise altered. In the same or a different embodiment, one or more of blocks 13100, 13260, 13300, 13400, 13450, 13500, 13600, and/or 13700 of method 13000 can comprise parts of a single block.

Although the invention has been described with reference to specific embodiments, it will be understood by those skilled in the art that various changes may be made without departing from the spirit or scope of the invention. For example, method 13000 of FIG. 13 can be expanded with further blocks. In one example, method 13000 could further comprise coupling a beamformer, such as beamformer 1400 (FIG. 1), with the first and second ultrasound arrays. In the same or a different example, method 13000 can further comprise coupling a scan converter, such as scan converter 1500 (FIG. 1), with the display of block 13200. In the same or a different example, method 13000 can further comprise incorporating the display of block 13200, and the first and second ultrasound arrays of blocks 13300 and 13400, with the housing of block 13100. In the same or a different example, method 13000 can further comprise providing a portable and/or rechargeable power source, such as power source 1700 (FIG. 1), coupled to the housing of block 13100. In the same or a different example, method 13000 can further comprise configuring the housing of block 13100 for single-handed and/or non-dominant-handed operation of the handheld imaging device, as described above for monitoring device 1000. Such alternate configurations would not depart from the inventive concepts herein disclosed. Additional examples have been given in the foregoing description.

Accordingly, the disclosure of embodiments of the invention is intended to be illustrative of the scope of the invention and is not intended to be limiting. It is intended that the scope of the invention shall be limited only to the extent required by the appended claims. To one of ordinary skill in the art, it will be readily apparent that the handheld imaging device and method for manufacture thereof discussed herein may be implemented in a variety of embodiments, and that the foregoing discussion of certain of these embodiments does not necessarily represent a complete description of all possible embodiments. Rather, the detailed description of the drawings, and the drawings themselves, disclose at least one preferred embodiment of the invention, and may disclose alternative embodiments of the invention.

All elements claimed in any particular claim are essential to the invention claimed in that particular claim. Consequently, replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

1. An ultrasound imaging device configured to facilitate subdermal monitoring; the ultrasound imaging device comprising:
a handheld housing;
a processor within the handheld housing;
a beamformer coupled to the processor;
a transducer assembly coupled to the handheld housing and to at least one of the beamformer or the processor;
a scan converter coupled to the transducer assembly;
a display coupled to the handheld housing and coupled to at least one of the scan converter or the processor;
a switch mechanism coupled to the processor;
a rechargeable power source coupled to the handheld housing;
a communications port coupled to the processor;
a central pointer aligned with a center of the display; and
a needle guide coupled to the handheld housing proximate to the transducer assembly;
wherein:
the transducer assembly comprises:
a first transducer array coupled to the processor and aligned along a first axis;
and
a second transducer array coupled to the processor and aligned along a second axis different from the first axis;
the first transducer array and the second transducer array are configured to produce different but overlapping scans of a target focus point;
the first axis is longitudinal to the target focus point;
the second axis is transverse to the target focus point;
the first transducer array and the second transducer array are substantially perpendicular to each other;
the first transducer array comprises transducer elements configured to scan images along the first axis;
the second transducer array comprises transducer elements configured to scan images along the second axis;
the first and second transducer arrays are capable of concurrently imaging the target focus point;
the rechargeable power source is cordless and configured to power the monitoring device uninterrupted for at least approximately a half-hour; and
the ultrasound imaging device is configured for single-handed operation.

2. The ultrasound imaging device of claim 1, further comprising:
a disposable casing comprising a transducer cover and configured to removably contain at least a portion of the handheld housing; and
a transparent gel-pack configured to couple with the disposable casing proximate to the transducer cover.

3. A cover for an ultrasound device having a display, a first transducer array aligned in a T-shape with a second transducer array, the cover comprising:
a casing configured to accommodate the T-shape; and
a gel-pack.

4. The cover of claim 3, wherein:
the cover is at least one of disposable or sterilizable.

5. The cover of claim 3, wherein:
at least a portion of the cover is transparent with regards to the first and second transducer arrays.

6. The cover of claim 3, wherein:
a thickness of the casing is between approximately 0.5 and 5 millimeters.

7. The cover of claim 3, wherein:
at least a portion of the casing is conformed to a shape of a portion of the ultrasound device.

8. The cover of claim 3, wherein:
the casing leaves the display exposed.

9. The cover of claim 3, further comprising:
a first portion between a second portion and a third portion,
wherein the first portion is configured to permit the second portion to be at least one of angled or rotated relative to the third portion.

10. The cover of claim 3, further comprising:
a non-stick material located at a portion of an exterior surface of the cover.

11. A monitoring device configured to facilitate intra-tissue inspection on a patient, the monitoring device comprising:
a housing;
a processor within the housing
a transducer coupled to the processor and to the housing;
at least one display coupled to the processor and to the housing;
wherein:
the transducer comprises:
a first transducer array coupled to the processor and aligned along a first axis;
and
a second transducer array coupled to the processor and aligned along a second axis different from the first axis;
the first transducer array and the second transducer array are configured to produce different but overlapping scans.

12. The monitoring device of claim 11, wherein:
the transducer is at least partially enclosed by the housing; and
the at least one display is integrated with the housing.

13. The monitoring device of claim 11, further comprising:
a beamformer coupled to the transducer;
wherein the processor couples to the transducer via the beamformer.

14. The monitoring device of claim 11, further comprising:
a scan converter coupled to the at least one display.

15. The monitoring device of claim 11, wherein:
the first and second transducer arrays are ultrasound transducer arrays.

16. The monitoring device of claim 11, wherein:
the first and second transducer arrays overlap substantially perpendicular to each other.

17. The monitoring device of claim 11, wherein:
the first axis is longitudinal to a target location;
the second axis is transverse to the target location; and
the at least one display is substantially parallel to the second axis.

18. The monitoring device of claim 11, wherein:
the transducer is configured to simultaneously scan:
(a) a first set of readings of a target location using at least a portion of the first transducer array; and
(b) a second set of readings of the target location using at least a portion of the second transducer array.

19. The monitoring device of claim 11, further comprising:
a switch mechanism coupled to the processor;
wherein the switch mechanism is configured to:
deactivate the second transducer array and activate the first transducer array in response to a first setting of the switch mechanism; and
deactivate the first transducer array and activate the second transducer array in response to a second setting of the switch mechanism.

20. The monitoring device of claim 19, wherein:
the at least one display is configured to:
present images correlated to a first set of readings from the first transducer array in response to a first setting of the switch mechanism; and
present images correlated to a second set of readings from the second transducer array in response to a second setting of the switch mechanism.

21. The monitoring device of claim 11, wherein:
the at least one display is configured to simultaneously present:
(a) images correlated to readings from the first transducer array on a first portion of the at least one display; and
(b) images correlated to readings from the second transducer array on a second portion of the at least one display.

22. The monitoring device of claim 11, further comprising:
a portable and rechargeable power source coupled to the housing.

23. The monitoring device of claim 11, wherein:
the monitoring device is configured for one-handed operation.

24. The monitoring device of claim 11, wherein:
the monitoring device is configured for nondominant-handed operation.

25. The monitoring device of claim 11, wherein:
at least one of the first or second transducer arrays is configured to scan a depth of field of up to approximately 10 cm.

26. The monitoring device of claim 11, wherein:
at least one of the first or second transducer arrays is configured to scan a span of up to approximately 4 to 5 cm.

27. The monitoring device of claim 11, wherein:
at least one of the first or second transducer arrays is configured to scan at a transducer frequency of approximately between 2 and 50 MHz.

28. The monitoring device of claim 11, wherein:
the at least one display comprises:
a width of approximately 3 to 8 cm; and
a height of approximately 2 to 5 cm.

29. The monitoring device of claim 11, further comprising:
a central pointer to indicate a center of an image shown on the at least one display and correlated to a centerline of at least one of the first or second transducer arrays.

30. The monitoring device of claim 11, further comprising:
one or more gridmarks on the housing and aligned along an axis substantially parallel to at least one of the first or second axes; and
one or more grid pointers to demarcate on the at least one display subdivisions correlated to the one or more gridmarks.

31. The monitoring device of claim 11, further comprising:
a needle guide aligned with the first transducer array and proximate to a central portion of the second transducer array.

32. The monitoring device of claim 31, wherein:
the needle guide further comprises a needle alignment groove.

33. The monitoring device of claim 11, further comprising:
a casing comprising a transducer cover and configured to removably envelop at least a portion of the housing;
wherein the transducer cover of the casing is transparent with respect to the first transducer array and the second transducer array.

34. The monitoring device of claim 33, wherein:
the transducer cover of the casing further comprises a gel-pack positioned proximate to the first and second transducer arrays.

35. The monitoring device of claim 33, wherein:
the casing is configured to be at least one of:
disposable; or
sterilizable.

36. The monitoring device of claim 11, wherein:
a weight of the monitoring device is between approximately 0.3 and 0.7 kilograms.

37. The monitoring device of claim 11, wherein:
the at least one display is configured to present a data entry screen; and
the data entry screen is configured to accept input from at least one of:
a touch-screen;
a keypad; or
a point and click mechanism.

38. A method of manufacturing a handheld imaging device, the method comprising:
providing a housing;
coupling a display to the housing;
providing a first ultrasound array to couple to the housing along a first axis; and
providing a second ultrasound array to:
couple to the housing along a second axis different from the first axis; and
scan a target in a different but overlapping manner with the first ultrasound array.

39. The method of claim 38, wherein:
providing the second ultrasound array further comprises selecting the second ultrasound array to be substantially normal to the first ultrasound array.

40. The method of claim 38, further comprising:
providing a switch mechanism coupled to the housing;
wherein:
the switch mechanism is configured to:
deactivate the second ultrasound array and activate the first ultrasound array in response to a first setting of the switch mechanism; and
deactivate the first ultrasound array and activate the second ultrasound array in response to a second setting of the switch mechanism; and
the display is configured to:
present images scanned from the first ultrasound array in response to the first setting of the switch mechanism; and
present images scanned from the second ultrasound array in response to the second setting of the switch mechanism.

41. The method of claim 38, further comprising:
providing a needle guide aligned with the first transducer array and proximate to a central portion of the second transducer array.

42. The method of claim 38, further comprising:
providing a disposable casing with a transducer cover and configured to removably contain at least a portion of the housing.

43. A monitoring device configured to facilitate intra-tissue inspection on a patient, the monitoring device comprising:
a housing;
a transducer coupled to the housing and comprising:
a first transducer portion configured to generate a first scan of a target area along a first axis; and
a second transducer portion configured to generate a second scan of the target area along a second axis different from the first axis;
at least one display coupled to the housing; and
a communications port coupled to the housing;
wherein:
the at least one display is configured to present images that correspond to the first and second scans of the target area from the first and second transducer portions; and
the communications port is configured to transmit information to or from the monitoring device.

44. The monitoring device of claim 43, wherein:
the communications port is configured to transmit the information to or from at least one of:
a computer; or
a database.

45. The monitoring device of claim 43, wherein:
the transducer is at least partially enclosed by the housing; and
the at least one display is integrated with the housing.

46. The monitoring device of claim 43, further comprising:
a switch mechanism coupled to the housing;
wherein the switch mechanism is configured to:
deactivate the second transducer array and activate the first transducer array in response to a first setting of the switch mechanism; and
deactivate the first transducer array and activate the second transducer array in response to a second setting of the switch mechanism.

47. The monitoring device of claim 43, further comprising:
a switch mechanism coupled to the housing;
wherein the switch mechanism is configured to toggle the at least one display between:
the images correlated to a first set of readings from the first transducer portion; and
the images correlated to a second set of readings from the second transducer portion.

48. The monitoring device of claim 43, wherein:
the at least one display is configured to simultaneously present:
(a) the images correlated to readings from the first transducer portion on a first portion of the at least one display; and
(b) the images correlated to readings from the second transducer portion on a second portion of the at least one display.

49. The monitoring device of claim 43, further comprising at least one of:
a central pointer to indicate a center of an image shown on the at least one display and correlated to a centerline of at least one of the first or second transducer portions;
one or more gridmarks on the housing and aligned along an axis substantially parallel to at least one of the first or second axes;
one or more grid pointers to demarcate on the at least one display subdivisions correlated to the one or more gridmarks;
a needle guide proximate to a central portion of the second transducer portion; or
a casing comprising a transducer cover transparent with respect to the first and second transducer portions and configured to removably envelop at least a portion of the housing.

50. The monitoring device of claim 43, wherein;
the monitoring device is configured for single-handed operation.

51. The monitoring device of claim 43, further comprising:
a processor; and
a beamformer coupled between the transducer and the processor.

52. The monitoring device of claim 43, further comprising:
a scan converter coupled to the at least one display.

53. The monitoring device of claim 43, wherein:
the housing comprises a first portion, a second portion, and a joint between the first and second portions;
the at least one display is coupled to the first portion of the housing;
the transducer is coupled to the second portion of the housing; and
the first and second portions of the housing are at least one of angleable or rotatable relative to each other via the joint.

54. The monitoring device of claim 11, wherein:
the housing comprises a first portion, a second portion, and a joint between the first and second portions;
the at least one display is coupled to the first portion of the housing;
the transducer is coupled to the second portion of the housing; and
the first and second portions of the housing are at least one of angleable or rotatable relative to each other via the joint.

55. The method of claim 38, wherein:
coupling the display to the housing comprises coupling the display to a first portion of the housing;
providing the first ultrasound array comprises coupling the first ultrasound array to a second portion of the housing;
providing the housing comprises:
providing a joint coupling the first portion of the housing to the second portion of the housing;
and
the joint is configured for adjusting the first and second portions of the housing relative to each other via at least one of:
providing an angle between the first and second portions of the housing; or
rotating the first and second portions of the housing relative to each other;
